(19) United States
(12) Patent Application Publication
(10) Pub. No.: US 2011/0137333 A1
(43) Pub. Date: Jun. 9, 2011

(54) EMBOLIC PROTECTION DEVICE

(75) Inventors: Kyle Hendrickson, Litchfield, MN (US); Allen Utke, Andover, MN (US); Benjamin Arcand, Minneapolis, MN (US); James Anderson, Fridley, MN (US)

(73) Assignee: Boston Scientific SciMed, Inc., Maple Grove, MN (US)

(21) Appl. No.: 12/631,453

(22) Filed: Dec. 4, 2009

(51) Int. Cl. A61F 2/01

(52) U.S. Cl. 606/200

(57) ABSTRACT

The disclosure describes an embolic filter and a support structure therefor.

### TECHNICAL FIELD

This disclosure relates generally to embolic filters and support structures therefor.

### BACKGROUND

Human blood vessels often become occluded or blocked by plaque, thrombi, other deposits, or material that reduce the blood carrying capacity of the vessel. Should the blockage occur at a critical place in the circulatory system, serious and permanent injury, and even death, can occur. To prevent this, some form of medical intervention is usually performed when significant occlusion is detected.

Several procedures are now used to open these stenosed or occluded blood vessels in a patient caused by the deposit of plaque or other material on the walls of the blood vessels. Angioplasty, for example, is a widely known procedure wherein an inflatable balloon is introduced into the occluded region. The balloon is inflated, dilating the occlusion, and thereby increasing the intraluminal diameter.

Another procedure is atherectomy. During atherectomy, a catheter is inserted into a narrowed artery to remove the matter occluding or narrowing the artery, i.e., fatty material. The catheter includes a rotating blade or cutter disposed in the tip thereof. Also located at the tip are an aperture and a balloon disposed on the opposite side of the catheter tip from the aperture. As the tip is placed in close proximity to the fatty material, the balloon is inflated to force the aperture into contact with the fatty material. When the blade is rotated, portions of the fatty material are shaved off and retained within the interior lumen of the catheter. This process is repeated until a sufficient amount of fatty material is removed and substantially normal blood flow is resumed.

In another procedure, stenosis within arteries and other blood vessels is treated by permanently or temporarily introducing a stent into the stenosed region to open the lumen of the vessel. The stent typically includes a substantially cylindrical tube or mesh sleeve made from such materials as stainless steel or nitinol. The design of the material permits the diameter of the stent to be radially expanded, while still providing sufficient rigidity such that the stent maintains its shape once it has been enlarged to a desired size.

Such percutaneous interventional procedures, i.e., angioplasty, atherectomy, and stenting, can dislodge material from the vessel walls. This dislodged material can enter the bloodstream. Some existing devices and technology use a filter for capturing the dislodged material from the bloodstream.

### SUMMARY

This disclosure pertains to an intraluminal filter. Such an intraluminal filter may include a guidewire, a filter element and first and second filter support structures where the first support structure comprises a distal first plurality of fingers having joined distal ends and free proximal ends and the second support structure comprises a proximal second plurality of fingers having joined proximal ends and free distal ends. The filter further comprises a porous filter material associated with at least the free proximal ends of the first plurality of fingers and a portion of the free distal ends of the second plurality of fingers, wherein the filter material is attached at least to the free distal ends of the second plurality of fingers and the joined proximal ends of the second plurality of fingers are disposed proximal of the free proximal ends of the first plurality of fingers.

In some embodiments, the filter material of the intraluminal filter includes a plurality of apertures sized to pass blood cells and to retain emboli, thrombi, and other debris. The filter material may be supported by a support framework comprising a first plurality of fingers lying generally adjacent to the filter membrane and a second separate plurality of fingers attached to the filter membrane at the distal ends of the second plurality of fingers such that the joined proximal ends of the second plurality of fingers are disposed proximal of the free proximal ends of the first plurality of fingers.

In other embodiments, the intraluminal filter comprising separate first and second pluralities of fingers supporting a filter membrane further comprises a containment structure including two or more apertures and an activation member having two positions. In the first position, the activation member engages the two or more apertures to hold the filter in a collapsed delivery state and in the second position the activation member is disengaged from at least one of the apertures, thereby releasing the filter to deploy.

### BRIEF DESCRIPTION OF DRAWINGS

FIG. 1 is a side view of an embodiment of an embolic filter.

FIG. 2 is an axial view of the embolic filter of FIG. 1 along 2-2.

FIG. 3 is a side view of an embodiment of an embolic filter.

### DETAILED DESCRIPTION

The following description should be read with reference to the drawings wherein like reference numerals indicate like elements throughout the several views. The drawings, which are not necessarily to scale, are not intended to limit the scope of the claimed invention. The detailed description and drawings illustrate example embodiments of the claimed invention.

All numbers are herein assumed to be modified by the term “about.” The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

The embolic filters disclosed herein can include a guidewire or tube and may be advanced through a blood vessel until they are positioned downstream of an obstruction to be treated. The filters are then deployed in various ways, such as by withdrawing a catheter or sheath in which they had been compressed or by releasing them from a containment structure.

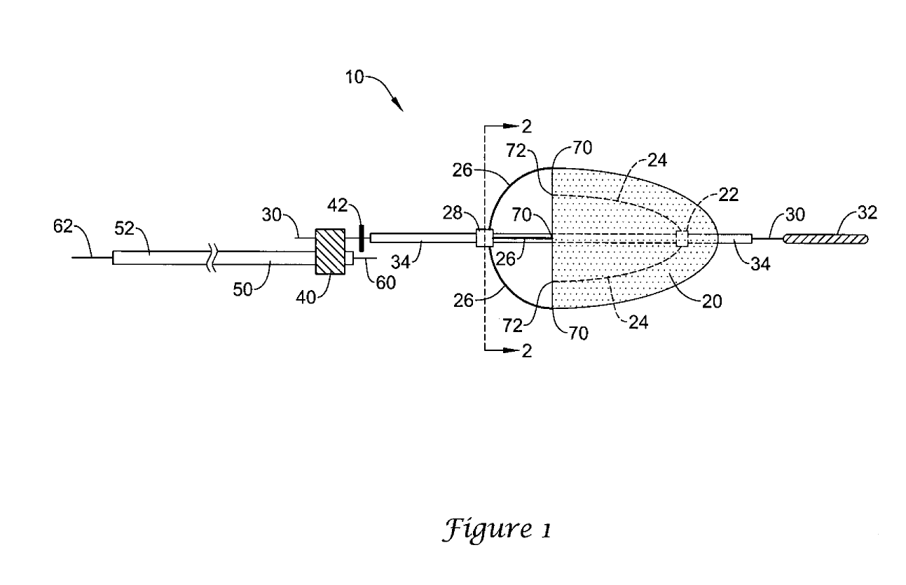

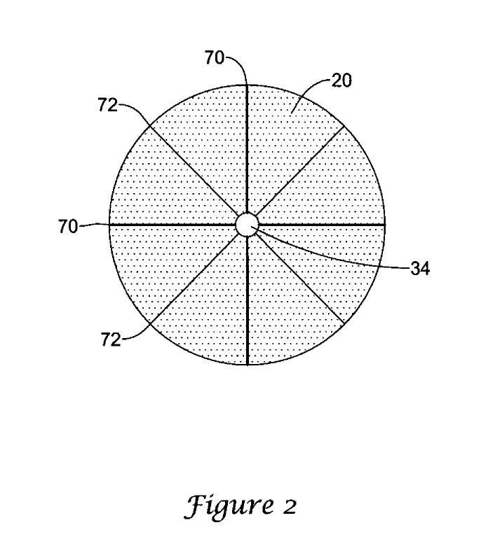

Turning to FIG. 1, filter basket 20 of filter assembly 10 has a generally conical or basket-like shape with a mouth that opens in the proximal direction. The filter basket or membrane 20 may be made of any of the materials commonly employed for that purpose including, but not limited to, perforated polymeric sheets and woven or nonwoven meshes. The filter material often includes a plurality of openings sized to pass blood cells, but to retain emboli, thrombi, and other debris. The filter basket 20 is supported by and between two pluralities of fingers 24,26. The first plurality of fingers 24 has distal ends which are joined at a hub 22 and free proximal ends which are not joined directly to other fingers. As used herein, “free ends” of fingers are those which are not directly connected to another finger at that end although the finger may be attached to a hub, ring, or other structure somewhat removed from the “free end” and a “free end” may optionally be attached to the filter basket 20. The second plurality of fingers 26 has proximal ends which are joined at hub 28 and free distal ends which are not joined directly to other fingers. As illustrated in the embodiment of FIG. 1, the first and second pluralities of fingers 24,26 are attached to filter basket 20 and attachment points 70,72 for the two pluralities of fingers alternate around the perimeter thereof as may be seen in the distal facing axial view presented in FIG. 2. The two pluralities are partially interdigitated; however they are not connected to each other at the mouth of the filter except indirectly through the filter basket 20 and any flexible reinforcing elements thereof. The method of attaching filter basket 20 to fingers 24,26 is within the ordinary skill of one in the art and may include, for example, adhesive bonding, solvent bonding, heat bonding, crimping, and the like. In the embodiment of FIG. 1, the distal hub 22 to which the first plurality of fingers 24 is attached is displaced somewhat proximally from the distal end of the filter basket 20 such that the fingers 24 are in contact with the filter basket 20 only near the mouth thereof.

Also in the embodiment of FIG. 1, hubs 22 and 28 are illustrated as disposed about an optional spinner tube 34 which in turn is disposed about guidewire segment 30. Spinner tube 34 allows rotation and limited translation of the deployed filter assembly 10 about guidewire segment 30 which is believed to minimize incidental damage to the lumen wall (not shown) as the guidewire segment 30 and extended hypotube 50 are manipulated during the time that the filter is deployed. The flexible guidewire tip 32 and optional stop 42 may be used as appropriate to limit axial travel of the spinner tube, upon which the filter is mounted, relative to guidewire segment 30. Although spinner tube 34 has been depicted as mounted on guidewire segment 30, it may also be mounted about an extended guidewire or hypotube 50 without intervening coupler 40. Coupler 40 provides a convenient offset to activation wire 60 when the filter assembly 10 is activated from a first contracted configuration to a second expanded configuration by means known in the art. Alternatively, the activation wire 60 may be used when the filter assembly 10 is released from within a containment structure by withdrawing the activation wire 60 from the containment structure (not shown here for clarity) by pulling proximal end 62 where it protrudes from proximal end 52 of hypotube 50. The offset may not be necessary if the filter assembly is self-expanding or if it is deployed from a sheath or delivery catheter instead of from a containment structure.

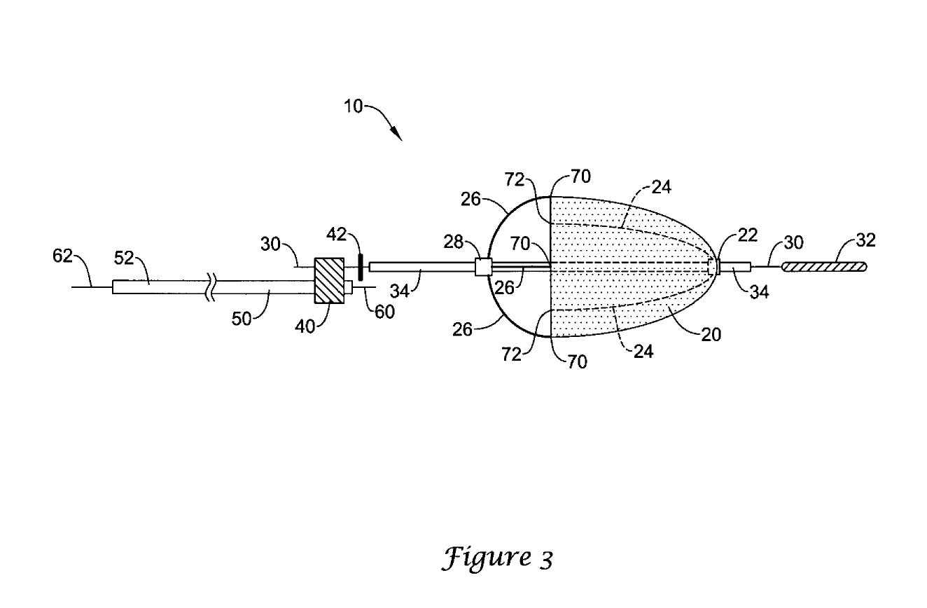

In the embodiment illustrated in FIG. 3, the distal hub 22, joining the first plurality of fingers 24 to optional spinner tube 34, is generally co-terminal with the filter basket 20 and the fingers 24 may lie along the inner surface of filter basket 20 and optionally may be attached thereto along their lengths to provide further definition to the shape of the filter basket 20.

In some embodiments, the filter basket material 20 is supported by a first and second plurality of fingers 24,26 wherein the first plurality of fingers generally lies within the basket 20 of the filter and supports the filter membrane forming the basket at least at the proximal tips of a portion of the fingers. In other embodiments, the first plurality of fingers may lie outside of the basket of the filter. The proximal tips of the first plurality of fingers 24 are free in the sense that they are not directly attached to the fingers of the second plurality of fingers 26 except through the filter basket 20 and any reinforcing material associated therewith. The proximal tips of the first plurality of fingers may be bonded to the filter material basket 20 or they may simply press outward on the filter material without being permanently attached thereto. In certain embodiments, a portion of the first plurality of fingers 24 may be bonded to the filter material and a portion of the fingers may remain unbonded. Such unbonded fingers may be longer or shorter than the bonded fingers. There may be groups of fingers having differing lengths within either the first and second pluralities of fingers or within both pluralities. Although the first plurality of fingers 24 need not be biased for outward expansion, some or all of the fingers may be so biased to assist in the deployment of the filter. The fingers of both pluralities of fingers may be formed from any of the polymeric, metallic, or composite materials commonly employed for filter supports; however it may be useful to employ shape-memory materials for those fingers which are to be outwardly biased. In addition to simple linear fingers, the fingers may be bifurcated, helical, or may be formed in any of a variety of configurations. The fingers may be round, oval, rectangular, hexagonal, or combinations of these and other cross-sectional shapes. The first and second pluralities of fingers 24,26 may, alone or in conjunction with other elements, form first and second support frameworks. In addition to fingers, support frameworks for the filter membrane may optionally include rings, spars, tethers, and the like to further shape and reinforce the filter. In some embodiments, the fingers within a first or second plurality of fingers need not all have the same composition. For example, the longer, tip bonded fingers of the first plurality of fingers may have a first composition while shorter, unbonded fingers may have a second composition.

In some embodiments, at least some the fingers of the first plurality of fingers 24 may be bonded to the filter material along their entire length and some may even be formed integrally with the filter material. Although the fingers of the first plurality of fingers are generally joined at a distal hub 22, in those embodiments in which the fingers are formed integrally with the filter material, the fingers may extend distally, but not join. For example, the fingers may taper distally until they are of zero thickness near the distal end of the filter basket formed by the filter material in which case they may be viewed as joined through the distal filter material. This is thought to facilitate folding the filter into a collapsed state suitable for delivery within a containment structure or delivery catheter. When the fingers of the first plurality of fingers are joined at a distal hub 22, the hub may be distally co-terminal with the filter material or it may lie within the filter basket or even distally outside of the filter basket. In some embodiments, the distal ends of some of the fingers of the first plurality of fingers may terminate differently than other distal ends of that plurality of fingers. For example, the fingers may terminate at more than one hub or a portion of the fingers may terminate at a hub while a second portion of the fingers tapers to zero thickness. In certain embodiments, a hub 22 formed at the distal end of a first plurality of fingers may be disposed about a spinner tube 34 associated with the filter. The hub 22 may be fixed to the spinner tube 34 or the hub may be free to slide along the spinner tube. If the spinner tube is not present, the hub may be fixed to a guidewire 30 or hypotube associated with the intraluminal filter or the hub may be free to slide along the guidewire or hypotube.

The fingers of the second plurality of fingers 26 generally are self-expanding or have an expanding mechanism associated therewith. Although the first plurality of fingers 24 need not be biased for outward expansion, some or all of the fingers may be so biased to assist in the deployment of the filter. The fingers of the second plurality of fingers 26 may be formed from any of the polymeric, metallic, or composite materials commonly employed for filter supports. It may be useful to employ shape-memory materials for fingers which are to be outwardly biased. As with the first plurality of fingers, the second plurality of fingers has free ends. The distal free ends are free in the sense that they are not directly attached to the fingers of the first plurality of fingers except through the filter basket 20 and any reinforcing material therein. The distal free ends of the second plurality of fingers 26 are bonded to the filter basket material 20 generally along the mouth of the filter basket or a skirt associated therewith which may form a proximal portion of the filter basket. The proximal ends of the second plurality of fingers may be joined to a hub 28. In certain embodiments, the hub 28 may be disposed about a spinner tube 34 or about a guidewire 30. When the spinner tube is present, it may be confined to the distal region of a guidewire 30 by one or more stops 42 and/or by a guidewire tip 32. The stops and/or guidewire tip may allow the filter limited translation and rotation. In those embodiments in which a hub, formed at the distal ends of at least some of the fingers of the first plurality of fingers, is fixed to a spinner tube 34 or guidewire 30, the hub 28 formed at the proximal ends of the second plurality of fingers 26 may slide along the spinner tube or guidewire. In those embodiments in which a hub 22, formed at the distal ends of at least some of the fingers of the first plurality of fingers 24, is free to slide along the spinner tube or guidewire, the hub 28 formed at the proximal ends of the second plurality of fingers 26 may be fixed to the spinner tube or guidewire. Although embodiments have been discussed in which fingers are joined at a hub which is in turn fixed to a spinner tube or guidewire, one of ordinary skill in the art will appreciate that the fingers maybe fixed directly to the spinner tube or guidewire without an intervening hub.

In some embodiments, at least some of the proximal free ends of the first plurality of fingers 24 are located proximally of the distal free ends of the second plurality of fingers 26. In other embodiments, at least some of the proximal free ends of the first plurality of fingers 24 are located distally of the distal ends of the second plurality of fingers 26. In some embodiments, fingers of the first and second pluralities of fingers are at least partially radially interspersed, i.e., one or more fingers of one plurality of fingers may be bonded to the filter material between a pair of fingers of the second plurality of fingers. In at least the simplest configurations, when viewed axially, the first and second pluralities of fingers will appear to alternate and to be offset from each other as one circumnavigates the mouth of the filter.

The filter material 20 may be any commonly employed for that purpose. It may be formed as a sheet or from a mesh. In some embodiments, it may be a molded article. It may include holes sized to pass common blood components such as red and white blood cells and yet retain emboli, thrombi, and other debris. The holes may be present in the material as formed, or they may be added later by laser drilling, punching, or the like. Some filter membranes may include skirts, bands, stiffeners, or other features which serve to improve the seal between the expanded filter and the lumen wall. In some embodiments, the proximal edge of the filter material may be scalloped to create a series of protrusions and recesses. Such scalloped edges may reduce the amount of material which initially must be introduced into a retrieval catheter or sheath when the filter is removed following use. Various attachment patterns between such scallops and the first and second pluralities of fingers are possible. For example, the first plurality of fingers 24 may be attached to the filter material 20 or a scalloped skirt thereof, which forms the mouth of the filter, at the proximal projections or at the recesses. In those embodiments in which the first plurality of fingers 24 is attached to proximal projections, the tips of the fingers may be curved somewhat toward the central axis of the filter to facilitate entry of the filter into a retrieval catheter or sheath (not shown). In other embodiments, the second plurality of fingers 26 may be attached to the filter material 20 at the proximal projections and may even extend distally beyond the recesses. In yet other embodiments, the first and second pluralities of fingers may alternate as they attach to the proximal projections. As described above, the fingers of either plurality may be attached to the interior or to the exterior surface of the filter material 20. In some embodiments, the filter material may be reinforced in a region which includes one or more proximal ends of the first plurality of fingers 24 and one or more distal ends of the second plurality of fingers 26. The filter membrane 20 may be present in the form of a hemisphere, basket, cone, or frustum, as well as other shapes, at the discretion of the filter designer.

Some filters of the invention may be deployed from a conventional delivery sheath and/or catheter while other embodiments include a containment structure having two or more apertures configured to engage an activation member having a first position and a second position, wherein the activation member engages the two or more apertures in the first position and is disengaged from at least one of the apertures in the second position allowing the filter to deploy.

Although the illustrative examples described above relate to an embodiment in which the open mouth of the filter is directed proximally and away from the guidewire tip within the lumen, a reversal of the filter components is also contemplated to provide a filter in which the open mouth of the filter is directed distally and toward the guidewire tip. In such an embodiment, the descriptive terms “distal”, “proximal”, and their derivative forms may be exchanged as they relate to the filter and associated components.

Various modifications and alterations of this invention will become apparent to those skilled in the art without departing from the scope and principles of this invention, and it should be understood that this invention is not to be unduly limited to the illustrative embodiments set forth hereinabove. All publications and patents are herein incorporated by reference to the same extent as if each individual publication or patent was specifically and individually indicated to be incorporated by reference.

What is claimed is:

1. An intraluminal filter comprising:
a first distally disposed plurality of fingers having joined distal ends and free proximal ends;
a second proximally disposed plurality of fingers having joined proximal ends and free distal ends; and
a porous filter material associated with at least the free proximal ends of the first plurality of fingers and a portion of the free distal ends of the second plurality of fingers, wherein the filter material is attached to the free distal ends of the second plurality of fingers,
further wherein the joined proximal ends of the second plurality of fingers are disposed proximal of the proximal free ends of the first plurality of fingers.

2. The intraluminal filter of claim 1, wherein the first plurality of fingers and the second plurality of fingers are at least partially interspersed at their respective free ends.

3. The intraluminal filter of claim 1, wherein at least some of the proximal free ends of the first plurality of fingers are located proximally of the distal ends of the second plurality of fingers.

4. The intraluminal filter of claim 1, wherein at least some of the proximal free ends of the first plurality of fingers are located distally of the distal ends of the second plurality of fingers.

5. The intraluminal filter of claim 1, wherein the first plurality of fingers is attached to the filter material along the majority of the length of at least some of the fingers.

6. The intraluminal filter of claim 5, wherein the first plurality of fingers is formed integrally with the filter material.

7. The intraluminal filter of claim 1, wherein the proximal ends of the first plurality of fingers are biased to expand outwardly.

8. The intraluminal filter of claim 1, wherein the distal ends of the second plurality of fingers are biased to expand outwardly.

9. The intraluminal filter of claim 1, wherein the first plurality of fingers is connected to the filter material only proximate the free proximal ends thereof and the second plurality of fingers is attached to the filter material only proximate the free distal ends thereof.

10. The intraluminal filter of claim 1, wherein the filter material extends to a position distal of the joined distal ends of the first plurality of fingers.

11. The intraluminal filter of claim 1, further comprising a spinner tube.

13. The intraluminal filter of claim 1, wherein the filter material is reinforced in a region which includes one or more free proximal ends of the first plurality of fingers and one or more free distal ends of the second plurality of fingers.

14. The intraluminal filter of claim 1, wherein at least some of the fingers of a plurality of fingers are of different length than the remaining fingers of that same plurality.

15. An intraluminal filter comprising:
a filter membrane, said membrane further comprising a plurality of apertures sized to pass blood cells and to retain emboli and thrombi;
a proximal first support framework comprising a first plurality of fingers associated with the filter membrane and lying generally adjacent to the filter membrane, wherein first plurality of fingers has joined distal ends and free proximal ends;
a distal second separate support framework comprising a second plurality of fingers attached to the filter membrane at the distal ends of said second plurality of fingers, wherein second plurality of fingers has joined proximal ends and free distal ends.

16. An intraluminal filter of claim 15, wherein the intraluminal filter further comprises a spinner tube.

17. An intraluminal filter of claim 16, wherein the distal ends of the first plurality of fingers are joined to the spinner tube.

18. An intraluminal filter of claim 16, wherein the distal ends of the first plurality of fingers are joined to a hub which is translatable along the spinner tube.

19. An intraluminal filter of claim 15, wherein the first support framework lies within a concave envelope formed by the filter membrane.

20. An intraluminal filter of claim 29, wherein at least some of the fingers of the first support framework and some of the fingers of the second support framework are attached to the filter membrane in a region where those fingers of the first support framework and those fingers of the second support framework overlap axially such that when viewed axially the fingers of the first support framework and the fingers of the second support framework appear offset relative to each other about a circumference of the filter material.
